(19) United States
(12) Patent Application Publication
(10) Pub. No.: US 2019/0329036 A1
(43) Pub. Date: Oct. 31, 2019

(54) MITIGATING UNEXPECTED SYNCOPE WITH VESTIBULAR STIMULATION

(71) Applicant: ICAHN SCHOOL OF MEDICINE AT MOUNT SINAI, new York, NY (US)

(72) Inventors: Bernard COHEN, New York, NY (US); Dmitri OGORODNIKOV, New York, NY (US)

(21) Appl. No.: 16/312,848

(22) PCT Filed: Jun. 23, 2017
(86) PCT No.: PCT/US17/39112

§ 371 Date: Dec. 21, 2018

Related U.S. Application Data
(60) Provisional application No. 62/354,602, filed on Jun. 24, 2016.

(51) Int. Cl. A61N 1/36

(52) U.S. Cl. CPC A61N 1/36036

(57) ABSTRACT

Resistance to the induction of a vasovagal response can be imparted in an animal by inducing galvanic vestibular stimulation of the animal and repeating the galvanic vestibular stimulation such that the animal is habituated to resist the induction of the vasovagal response.

### BACKGROUND

Vasovagal syncope (VVS) is a significant medical problem. The symptoms that lead to a VVS and the preceding VasoVagal Response (VVR) that underlies the syncope have been well described, and the reductions in baroreflex sensitivity and in blood pressure (BP) and heart rate (HR) that are associated with syncope are also known. In studies of combined tilt and Lower Body Negative Pressure, BP fell, HR transiently increased, but then also rapidly declined in the pre-syncopal state. An important observation was that the VVR and VVS involved a reduction in baroreflex sensitivity. Why this occurs is still unknown. Consequently, there has been no effective therapy for VasoVagal Syncope. Therapeutic measures have included beta blockers, corticosteroids, and pacemakers, but none of these has been generally more effective than placebo.

An apparently promising therapy in which ‘syncope-sensitive’ patients were repetitively tilted up 60° for prolonged periods was originally shown to habituate the VSR and reduce or abolish syncope, using static Head-up Tilt that activated otolith and Body Tilt Receptors (BTRs), which play a major role in producing cardiovascular changes through the VSR. Sustained habituation of syncope was not found in other studies that utilized tilt training, however. It has been speculated that although it was probably possible to habituate some subjects with prolonged bouts of static head-up tilt, the habituating techniques were too tedious to be effective. If a less tedious procedure were devised to habituate syncope through the VSR, however, it could be used to reduce or block VVRs and VVS in humans.

The following publications are incorporated herein by reference for all purposes.
1. Raphan, T., A parameter adaptive approach to oculomotor system modeling, in Engineering. 1976, CUNY: New York. p. 204.
2. Gendelman, H. E., et al., Syncope in a general hospital patient population. Usefulness of the radionuclide brain scan, electroencephalogram, and 24-hour Holter monitor. N Y State J Med, 1983. 83(11-12): p. 1161-1165.
3. Soteriades, E. S., et al., Incidence and prognosis of syncope. N Engl J Med, 2002. 347(12): p. 878-85.
4. Grubb, B., P., Neurocardigenic syncope. The New England Journal of Medicine, 2005. 352: p. 1004-1010.
5. Julu, P. O. O., et al., Cardiovascular regulation in the period preceding vasovagal syncope in conscious humans. J. Physiol., 2003. 549 (1): p. 299-311.
6. Moya, A., et al., Guidelines for the diagnosis and management of syncope (version 2009). Task Force for the Diagnosis and Management of Syncope; European Society of Cardiology (ESC); European Heart Rhythm Association (EHRA); Heart Failure Association (HFA); Heart Rhythm Society (HRS). Eur Heart J, 2009. 30 (21): p. 2631-2671.
7. Lewis, T., Vasovagal syncope and the carotid sinus mechanism. Br Med J, 1932. 3723 (1): p. 873-876.
8. Thomson, H. L., K. Wright, and M. Frenneaux, Baroreflex sensitivity in patients with vasovagal syncope. Circulation, 1997. 95 (2): p. 395-400.
9. Kaufmann, H. and R. Hainsworth, Why do we faint? Muscle Nerve, 2001. 24: p. 981-983.
10. Morillo, C. A. and J. C. Villar, Neurocardiology. Neurogenic syncope. Baillieres Clin Neurol, 1997. 6 (2): p. 357-80.
11. Mosqueda-Garcia, R., et al., Sympathetic and baroreceptor reflex function in neurally mediated syncope evoked by tilt. J Clin Invest, 1997. 99 (11): p. 2736-2744.
12. Calkins, H., Pharmacological approaches to therapy for vasovagal syncope. Am. J. Cardiol, 1999. 84: p. 20Q-25Q.
13. Sheldon, R., S. Connolly, and S. Vasovagal Pacemaker, II, Second Vasovagal Pacemaker Study (VPS II): rationale, design, results, and implications for practice and future clinical trials. Card Electrophysiol Rev, 2003. 7 (4): p. 411-5.
14. Sheldon, R., S. Rose, and S. Connolly, Prevention of Syncope Trial (POST): a randomized clinical trial of beta blockers in the prevention of vasovagal syncope; rationale and study design. Europace, 2003. 5 (1): p. 71-5.
15. Ector, H., et al., Tilt training: a new treatment for recurrent neurocardiogenic syncope and severe orthostatic intolerance. Pacing Clin Electrophysiol, 1998. 21 (1 Pt 2): p. 193-6.
16. Di Girolamo, E., et al., Effects of paroxetine hydrochloride, a selective serotonin reuptake inhibitor, on refractory vasovagal syncope: a randomized, double-blind, placebo-controlled study. J Am Coll Cardiol, 1999. 33(5): p. 1227-1230.
17. Reybrouck, T., et al., Long-term follow-up results of tilt training therapy in patients with recurrent neurocardiogenic syncope. Pacing Clin Electrophysiol, 2002. 25 (10): p. 1441-6.
18. Kinay, O., et al., Tilt training for recurrent neurocardiogenic syncope: effectiveness, patient compliance, and scheduling the frequency of training sessions. Jpn Heart J, 2004. 45 (5): p. 833-43.
19. Yates, B. J., M. J. Holmes, and B. J. Jian, Adaptive plasticity in vestibular influences on cardiovascular control. Brain Res Bull, 2000. 53 (1): p. 3-9.
20. Yates, B. J., et al., Responses of vestibular nucleus neurons to tilt following chronic bilateral removal of vestibular inputs. Exp Brain Res, 2000. 130 (2): p. 151-158.
21. Yates, B. J., et al., Organization of vestibular inputs to nucleus tractus solitarius and adjacent structures in cat brain stem. Am J Physiol, 1994. 267 (4 Pt 2): p. R974-R983.
22. Yates, B. J. and A. D. Miller, Properties of sympathetic reflexes elicited by natural vestibular stimulation: implications for cardiovascular control. J Neurophysiol, 1994. 71 (6): p. 2087-2092.
23. Cohen, B., et al., Sinusoidal galvanic vestibular stimulation (sGVS) induces a vasovagal response in the rat. Exp Brain Res, 2011. 210 (1): p. 45-55.
24. Cohen, B., S. B. Yakushin, and G. R. Holstein, What does galvanic vestibular stimulation actually activate? Front Neurol, 2012. 2 (90): p. 2:90. doi: 10.3389/fneur.2011.00090.
25. Holstein, G. R., V. L. Friedrich, Jr., and G. P. Martinelli, Projection neurons of the vestibulo-sympathetic reflex pathway. J Comp Neurol, 2014. 522 (9): p. 2053-74.
26. Holstein, G. R., et al., Fos expression in neurons of the rat vestibulo-autonomic pathway activated by sinusoidal galvanic vestibular stimulation. Front Neurol, 2012. 3 (4): p. doi: 10.3389/fneur.2012.00004.
27. Yates, B. J., P. S. Bolton, and V. G. Macefield, Vestibulo-sympathetic responses. Comprehensive Physiology, 2014. 4 (2): p. 851-887.
28. Foglia-Manzillo, G., et al., Efficacy of tilt training in the treatment of neurally mediated syncope. A randomized study. Europace, 2004. 6 (3): p. 199-204.
29. Duygu, H., et al., The role of tilt training in preventing recurrent syncope in patients with vasovagal syncope: a prospective and randomized study. Pacing Clin Electrophysiol, 2008. 31 (5): p. 592-6.
30. Nowak, J. A., et al., Multiresolution wavelet analysis of time dependent physiological response in syncopal youths. Am. J. Physiol. Heart Circ. Physiol., 2009. 296: p. H171-H179.
31. Cohen, B., et al., The vaso-vagal response (VVR) of the rat: its relation to the vestibulo-sympathetic reflex (VSR) and to Mayer waves. FASEB, 2013. 27 (7): p. 2564-2572.
32. Yakushin, S. B., et al., Vasovagal oscillations and vasovagal responses produces by the vestibulo-sympathetic reflex in the rat. Frontiers in Neurology, 2014. 5: p. 37-.
33. Bolton, P. S., D. L. Wardman, and V. G. Macefield, Absence of short-term vestibular modulation of muscle sympathetic outflow, assessed by brief galvanic vestibular stimulation in awake human subjects. Exp. Brain Res., 2004. 154 (1): p. 39-43.
34. Grewal, T., C. James, and V. G. Macefield, Frequency-dependent modulation of muscle sympathetic nerve activity by sinusoidal galvanic vestibular stimulation in human subjects. Exp Brain Res, 2009. 197 (4): p. 379-86.
35. Hammam, E., T. Dawood, and V. G. Macefield, Low-frequency galvanic vestibular stimulation evokes two peaks of modulation in skin sympathetic nerve activity. Exp Brain Res, 2012. 219 (4): p. 441-6.
36. Klingberg, D., E. Hammam, and V. G. Macefield, Motion sickness is associated with an increase in vestibular moulation of skin but not muscle sympathetic nerve activity. Exp. Brain Res., 2015. 233 (8): p. 2433-2440.
37. Yakushin, S. B., et al., The response of the vestibulosympathetic reflex to linear acceleration in the rat. Frontiers in Neuroscience—Systems Neuroscience, 2016 (In review).
38. Raphan, T., et al., A model of blood pressure, heart rate, and vaso-vagal responses produced by vestibulo-sympathetic activation. Frontiers in Neuroscience—Autonomic Neuroscience, 2016 (In press).
39. Fredrickson, J. and H. Kornhuber. The cortical projection of the vestibular nerve in the Rhesus monkey. in International Symposium on Vestibular and Oculomotor Problems. 1965. Japan Society of Vestibular Research, University of Tokyo, Tokyo, Japan.
40. Mittelstaedt, H., Somatic graviception. Biol Psychol, 1996. 42 (1-2): p. 53-74.
41. Mittelstaedt, H., Origin and processing of postural information. Neuroscience & Biobehavioral Reviews, 1998. 22 (4): p. 473-478.
42. Yavorcik, K. J., et al., Effects of postural changes and removal of vestibular inputs on blood flow to and from the hindlimb of conscious felines. Am J Physiol Regul Integr Comp Physiol, 2009. 297 (6): p. R1777-R1784.
43. Barcroft, H. and O. G. Edholm, On the vasodilation in human skeletal muscle during post-haemorrhagic fainting. J. Physiol., 1945. 104.2: p. 161-175.
44. Kaufmann, H., et al., Vestibular control of sympathetic activity: An otolith-sympathetic reflex in humans. Exp. Brain Res., 2002. 143: p. 463-469.
45. Julien, C., The enigma ofMayer waves: Facts and models. Cardiovascular Research, 2006. 70: p. 12-21.
46. Mayer, S., Studien zur Physiologic des Herzens and der Blutgefässe. Sitz Kaiser Akad Wiss, 1876. 74: p. 281-307.
47. Xiang, Y., et al., Dynamics of quadrupedal locomotion of monkeys: implications for central control. Exp. Brain Res., 2006.
58. Osaki, Y., et al., Relative contribution of walking velocity and stepping frequency to the neural control of locomotion. Exp. Brain Res., 2008. 185: p. 121-135.
59. Berne, R. M. and M. N. Levy, Cardiovascular physiology. 2001, Philadelphia, Pa.: Mosby.
6ork, NY: McGraw-Hill.
61. Blessing, W. W., The lower brainstem and bodily homeostasis. 1997: Oxford University Press.
62. Granata, A. R., Modulatory inputs on sympathetic neurons in the rostral ventrlateral medulla in the rat. Cellular and Molecular Neurobiology, 2003. 23(4/5): p. 665-680.
63. Barcroft, H., et al., Posthaemorrhagic fainting: Study by cardiac output and forearm flow. The Lancet, 1944: p. 489-490.
64. Tsubota, T., et al., Optogenetic inhibition of Purkinje cell activity reveals cerebellar control of blood pressure during postural alterations in anesthetized rats. Neuroscience, 2012. 210: p. 137-144.
65. Bradley, D. J., et al., An electrophysiological and anatomical study of afferents reaching the cerebellar uvula in the rabbit. Experimental Physiology, 1990. 75: p. 163-177.
66. Paton, J. F. R., et al., Efferent connections of lobule IX of the posterior cerebellar cortex in the rabbit—some functional considerations. Journal of the Autonomic Nervous System, 1991. 36: p. 209-224.
67. Doba, N. and D. J. Reis, Changes in regional blood flow and cardiodynamics evoked by electrical stimulation of the fastigial nucleus in the cat and their similarity to orthstatic reflexes. Circ. Res., 1974. 34: p. 9-18.
68. Pan, P. S., Y. S. Zhang, and Y. Z. Chen, [Role of the nucleus vestibularis medialis in vestibulo-sympathetic response in rats]. Acta Physiol Sin, Chinese, 1991. 43 (2): p. 184-188.
69. Uchino, Y., et al., Vestibular inhibition of sympathetic nerve activities. Brain Res Bull, 1970. 22 (2): p. 195-206.
70. Yates, B. J., T. Goto, and P.S. Bolton, Responses of neurons in the rostral ventrolateral medulla of the cat to natural vestibular stimulation. Brain Res, 1993. 601 (1-2): p. 255-264.
71. Yates, B. J., Vestibular influences on the sympathetic nervous system. Brain Res, 1992. 17 (1): p. 51-59.
72. Bozdagi, O., et al., Imidazoleacetic acid-ribotide induces depression of synaptic responses in hippocampus through activation of imidazoline receptors. J Neurophysiol, 2011. 105 (3): p. 1266-1275.
73. Martinelli, G. P., et al., Vestibular neurons in the rat contain imidazoleacetic acid-ribotide, a putative neurotransmitter involved in blood pressure regulation. J Comp Neurol, 2007. 501 (4): p. 568-581.
74. Prell, G. D., et al., Imidazoleacetic acid-ribotide: an endogenous ligand that stimulates imidazol(in)e receptors. Proc Natl Acad Sci, 2004. 101 (37): p. 13677-13682.
75. Davos, C. H., L. C. Davies, and M. Piepoli, The effect of baroreceptor activity on cardiovascular regulation. Hellenic J. Cardiol, 2002. 43: p. 145-155.
76. La Rovere, M. and G. Pinna, Raczak, G., Baroreflex sensitivity: measurement and clinical implications. Ann Noninv Electrocardio 01, 2008. 13 (2): p. 191-207.
77. Osaki, Y., et al., Three dimensional kinematics and dynamics of the foot during walking: a model of central control mechanisms. Exp. Brain Res., 2007. 176: p. 476-496.
78. Cho, C., et al. A model-based approach for assessing Parkinsonian gait and effects of levadopa and deep brain stimulation. in Proceedings of the 28th IEEE, EMBS Annual International Conference. 2006. New York City.
79. Cho, C., et al., Frequency-velocity mismatch: A fundamental abnormality in Parkinsonian gait. J. Neurophysiol., 2010. 103: p. 1478-1489.
80. Goldberg, J. M., C. Fernandez, and C. E. Smith. Responses of vestibular nerve afferents in the squirrel monkey to externally applied galvanic currents. Brain Res., 1982, 252 (1): 156-160, 1982.
81. Goldberg, J. M., C. E. Smith, and C. Fernandez. Relation between discharge regularity and responses to externally applied galvanic currents in vestibular nerve afferents of the squirrel monkey. J. Neurophysiol., 1984, 51 (6): 1236-1256.

### BRIEF SUMMARY

In accordance with at least one embodiment of the present disclosure, a method of imparting resistance to vasovagal response in a patient is disclosed. The method includes inducing galvanic vestibular stimulation of the patient, e.g. of the vestibular system in general, or of the vestibular otolith in particular; and repeating the inducing galvanic vestibular stimulation such that the patient is habituated to resist an induction of the vasovagal response. According to various embodiments, the method includes inducing galvanic vestibular stimulation via an alternating current such that the alternating current stimulates a vestibular otolith of the patient, which may include applying a repeating waveform at a treatment amplitude in the range of 0.4 to 2 mA, and having a frequency in the range of 0.01 Hz to 0.05 Hz. The waveform can be any suitable repeating waveform, but in specific embodiments, can be a sinusoidal waveform. The galvanic vestibular stimulation can be induced by applying the stimulation via one or more electrodes at a mastoidal site of the patient such that the stimulation is applied to a vestibular otolith.

According to some embodiments, each individual treatment can include inducing the galvanic vestibular stimulation for a first ramp period and a second treatment period after the ramp period, ramping from an amplitude of 0 to a treatment amplitude during the ramp period, and inducing the stimulation at the treatment amplitude for the second treatment period. The ramp period can have a duration of at least 200 seconds, e.g. from 200 to 300 seconds, or more. A total treatment can include applying the stimulation for at least one period of time corresponding to 15 to 30 minutes, and repeating the stimulation any suitable number of times until resistance to the vasovagal response is achieved, e.g., once, two or more times, three or more times, etc. According to some embodiments, the particular form of vasovagal response treated is unexpected syncope.

In accordance with at least one embodiment of the present disclosure, a system for imparting resistance to vasovagal response in a patient by galvanic stimulation is disclosed. The system can include a controller including a processor and nontransitory memory containing executable instructions that, when executed by the processor, cause the controller to generate a repetitive waveform, a current driver operably connected with the controller and configured to generate a signal suitable for application to a patient based on the waveform; and at least one pair of electrical leads operably connected with the current driver and configured to impart the signal to the patient when the pair of electrical leads is connected to the patient at a treatment site corresponding with the vestibular system of the patient. According to some embodiments, the electrical leads are electrodes that attach to the skin of a patient proximate to a mastoidal site, which may be configured to stimulate the vestibular otolith of the patient.

In accordance with some embodiments, the system can include an isolation element operably connected between the controller and the current driver. According to some embodiments, the system can also include a second controller operably connected between the isolation element and the current driver, where the second controller generates a second waveform based on the first waveform filtered by the isolation element, and the current driver is configured to generate the signal suitable for application to a patient based on the second waveform. The controller can be further configured to generate the repetitive waveform, which can be a sinusoidal waveform or any other, similar, form of suitable repeating waveform, at a frequency in the range of 0.01 to 0.05 Hz, and/or at a frequency of about 0.025 Hz. According to some embodiments, the controller is also configured to generate the waveform at an increasing amplitude for a first nonzero duration of a ramp period during which the amplitude increases from an initial amplitude to a treatment amplitude, and to generate the waveform at the treatment amplitude for a second nonzero duration of a treatment period subsequent to the ramp period. The ramp period can be at least 200 seconds, at least 300 seconds, or in some cases can range from 200 seconds to 300 seconds. The subsequent treatment period can have a duration in the range of 20 to 30 minutes.

### BRIEF DESCRIPTION OF THE DRAWINGS

FIGS. 1A-1B show habituation with sGVS: Incidence of VasoVagal Responses (VVRs) on successive test days. FIG. 1A shows a susceptible rat that had VVRs on every test on the first day (100%). Animal successively became resistant as testing continued over 12 test days and a month. Initially, the rat had a VVR on each test and by the 5^{th} day, similar stimuli did not induce any VVRS. Resistance to induction of VVRs continued throughout subsequent testing to the 12^{th} day. FIG. 1B shows reduction in susceptibility in 8 susceptible rats. Initially, the rats had VVRs over 80% when stimulated, which then decreased until there were no responses only on the 10^{th} day and then no responses on the 12^{th} day. Percentage of VVR induction is shown on the ordinate and he test day on the abscissa.

FIG. 2 shows Habituation with oscillation in Pitch: Schema

FIGS. 3A-3F show BP and HR changes during habituation, as follows: FIG. 3A: In response to a 70 degree nose-up tilt (3d trace), BP (Top Trace) and HR (2nd Trace) both fell producing a VVR. FIG. 3D: Similar drops in BP and HR were produced by sGVS. FIGS. 3B, 3E: With repeated habituation, BP still fell when the VSR was activated, but HR now rose opposing the fall in BP. FIGS. 3C, 3F Finally, both BP and HR rose slightly when there was VSR stimulation and VVRs could not be induced.

FIGS. 4A-4B show a rise in Baroreflex Sensitivity (BRS) with habituation (FIG. 4A) and incidence of 0.025 oscillations in BP in a VVR susceptible rat. After prolonged activation with sGVS, the low frequency activation of BP disappeared (FIG. 4B). Low frequency activation of HR also disappeared (not shown).

FIG. 5 shows a simplified block diagram illustrating an example system for inducing galvanic vestibular stimulation in a patient, in accordance with embodiments.

### DETAILED DESCRIPTION

In the following description, various embodiments of the present invention will be described. For purposes of explanation, specific configurations and details are set forth in order to provide a thorough understanding of the embodiments. However, it will also be apparent to one skilled in the art that the present invention may be practiced without the specific details. Furthermore, well-known features may be omitted or simplified in order not to obscure the embodiment being described.

Abbreviations: BP—arterial blood pressure; BRS—Baroreflex Sensitivity; BTR—Body Tilt Receptor; g—acceleration of gravity; HR—heart rate, beats/min; MSNA—Muscle Sympathetic Nerve Activity; RVLM—Rostral Ventral Lateral Medulla;; sGVS—sinusoidal Galvanic Vestibular Stimulation; VSR—VestibuloSympathetic Reflex; VVR—VasoVagal Response; VVS—VasoVagal Syncope.

Definitions: Baroreflex Sensitivity—Measure of average changes in intersystolic interval due to changes in systolic BP; Body Tilt Receptor—Somatosensory input to the Vestibular Nuclei that sense body position re the direction of gravity; Susceptible rat—A rat that readily develops VVRs after being stimulated with sGVS and tilts when anesthetized. Non-susceptible rat—rat that doesn't develop VVRs in response to sGVS and tilts.

Applicants have previously recognized that the anesthetized rat could be a small animal model of cardiovascular changes during VVRs. Many such susceptible rats readily developed synchronous ≈20-40 mmHg decreases in BP and ≈20-40 bpm decreases in HR that recover slowly. The decreases in BP and HR and the slower return to pre-stimulus values are important components of VVRs that underlie VasoVagal Syncope (VVS), but a finer discrimination of what is and what is not a VVR needed to be developed and is proposed in this application. A wide range of vestibular (otolith) stimuli, including those that generate linear accelerations along the Z-axis of the head and body are capable of inducing VVRs, including sinusoidal Galvanic Vestibular Stimulation (sGVS), translation while rotating, ±70° oscillation in pitch and 70° head-up tilt.

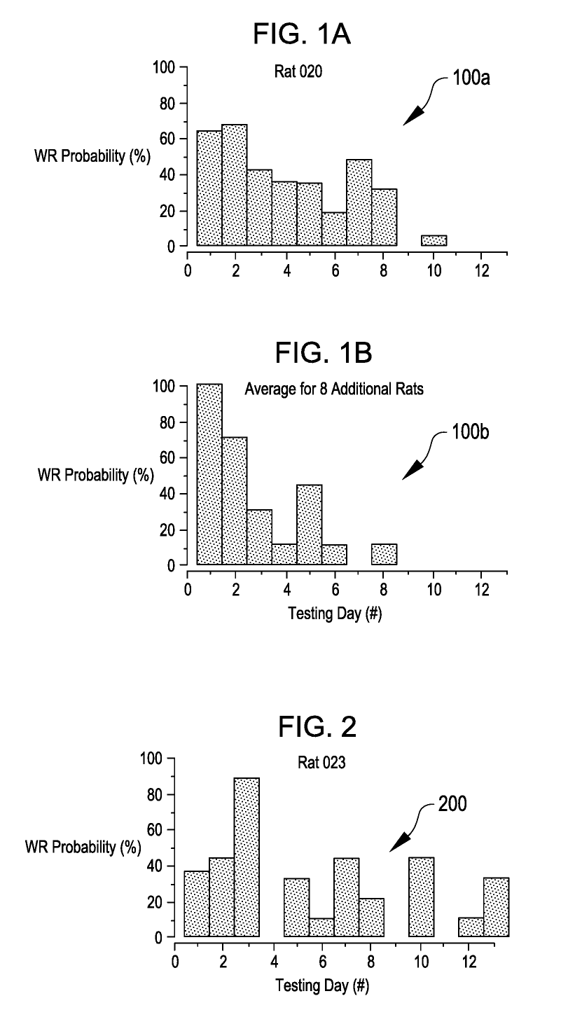

A striking finding was that all of the rats that were initially susceptible to induction of VVRs progressively lost their sensitivity as testing continued (Preliminary Data, FIG. 1A-1B). Among the susceptible rats that have been studied, none had an increase in susceptibility when tested regularly, although long cessation of testing could cause transient increases in susceptibility. When the animals were retested, however, the increased sensitivity again disappeared. Thus, the rats were becoming progressively unresponsive to the vestibular stimuli, i.e., that they were being habituated by the recurrent stimulation of the VSR. Similar habituation was also produced by ±70° oscillation in pitch, although pitch was less effective than sGVS as a habituating stimulus (FIG. 2). sGVS has been widely used in humans to activate MSNA), and is safe and harmless. If habituation was easily induced by activation of the VSR in rats, and if the underlying mechanisms of habituation were also known, applicants believed that such habituation could potentially be used to reduce the susceptibility of humans prone to Syncope.

This research produced increased understanding of the trigger mechanism that initiates the fall in BP and HR, and offers a possible technique to reduce occurrence of VVRs and VVSs in humans.

The small animal model of VVRs has important advantages. It is possible to test and retest rats and explore new hypotheses at length easily and without grief. This cannot always be done with humans.

Recurrent syncope could be habituated in 4 human studies, showing that this is possible, but was not found in 2 other studies. The habituating technique was believed to be too tedious. SGVS is simple to apply, is safe, and is used widely in studies of MSNA, and could be an effective technique for such habituation.

Identification of the anesthetized rat as a small animal model of the VasoVagal Response was an important innovation. It has enabled new research in this area and we can induce VVRs almost at will to study the processes resulting in the drop in BP and HR that underlie VVRs and VVS.

The finding that VVRs can be reduced along with dramatic changes in BP and HR provide a mechanism for potentially blocking VVRs and VVSs in humans.

Characterizing the Vestibulo-Sympathetic Reflex (VSR), and studying the changes in blood pressure (BP) and heart rate (HR):

Two publications showed that VVRs are only produced when low frequency oscillations (0.025 & 0.05 Hz) were present in BP and HR in the anesthetized rats during sGVS or when the rats were accelerated up or forward, and decreases in BP when they were moved downward. HR was not altered by these translations, and there was no response to lateral or backward translation. The VSR was shown to have low frequency characteristics. Thus, the VSR can be defined as the response to activation of a projection from the vestibular (Otolith) system to the cardiovascular system, altering MSNA and BP in humans and BP in rats in response to linear acceleration along the gravitational axis and forward. The absence of HR changes demonstrates that the VSR is processed through a separate component of the sympathetic system, which in normal humans produces increases in BP upon arising, (orthostasis) to sustain blood flow to the brain.

### Experimental Results

There was a progressive loss of susceptibility to induction of VVRs in test subjects (rats) after repeated exposure to ±2 mA, 0.025 Hz sGVS (FIG. 1A) and a similar loss of susceptibility in 8 other rats (FIG. 1B). With repeated testing, the animals had fewer VVRs until the VVRs could no longer be induced.

Habituation was also induced with ±70° oscillation in pitch but the susceptibility of individual rats varied and most rats required longer periods of habituation than with sGVS (FIG. 2).

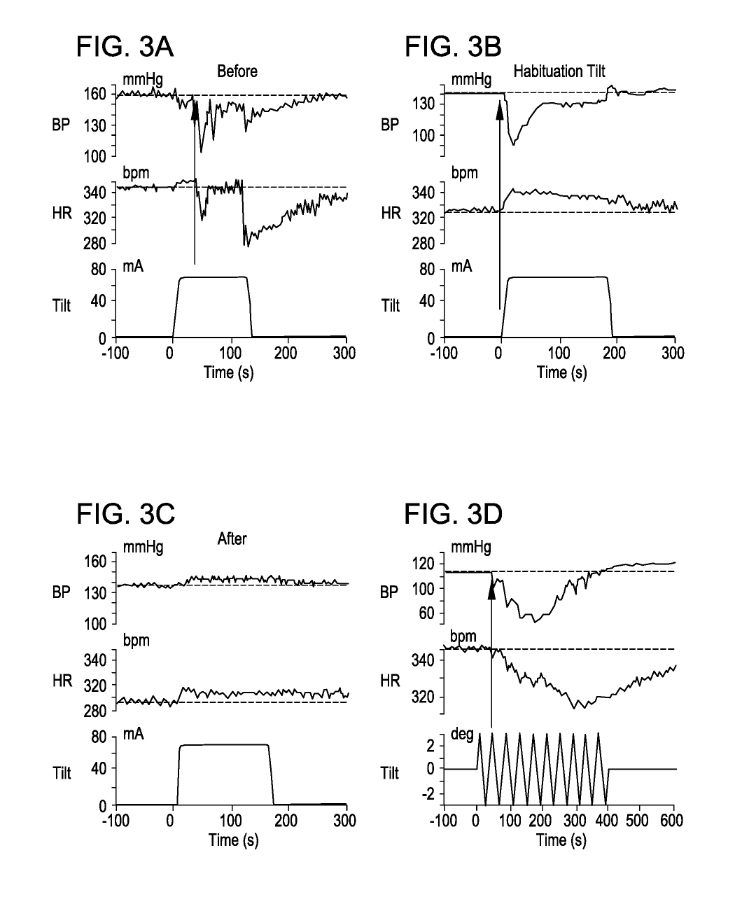

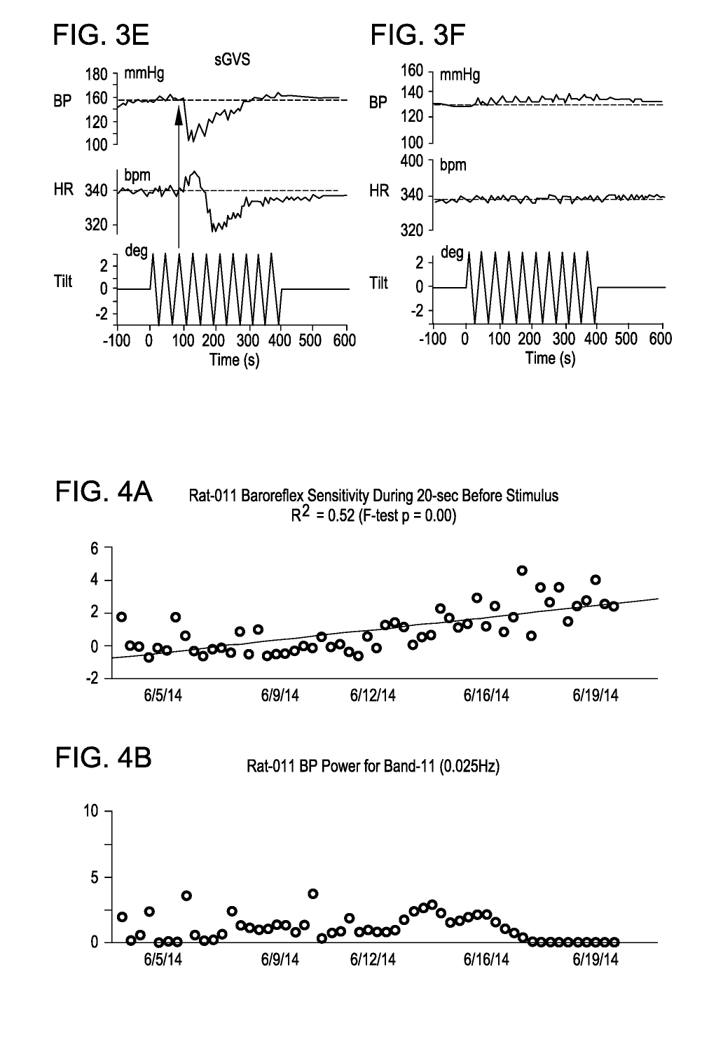

The change in susceptibility from the original state that generated VVRs (FIG. 3A, D) had an increase in HR to oppose drops in BP (FIG. 3B). There was a small increase in HR initially in response to the sGVS (FIG. 3E), but it was not sustained. Finally only small increases in BP and HR were induced when the VSR was activated (FIG. 3C, F). Only 3 examples are shown for each mode of stimulation (FIG. 3), but there was a steady progression in the development of the HR that opposed the drops in BP from the beginning to the end of habituation. This implied that there had been an increase in baroreflex sensitivity (BRS). The BRS was initially depressed under anesthesia (FIG. 4, top trace), but with continued activation of the VSR with sGVS, there was a steady rise in BRS throughout the period of habituation (FIG. 4A). The rise in BRS was significant (R²=0.52; p<0.01). Associated with this rise, there was a loss of the low frequency 0.025 Hz oscillations in BP (FIG. 4B) when complete habituation was attained (FIG. 4B). There was also a similar loss of low frequency activity in HR (not shown). Thus, the habituation could be characterized initially by a steady rise in HR which then subsided to a low level accompanied by a small rise in BP likely produced by the changes in BRS and a loss in the low frequency activity in BP and HR. The rise in BRS was probably related to the modification of HR and the loss of the low frequency oscillations in BP and HR but the source of the signal that drove the change in BRS is still not known.

### Experiment 1: Habituation With sGVS

### Experimental Protocol

During experiments, the animals were anesthetized with isoflurene, (Methods 1), and lay prone in a container. BP and HR were recorded by an adjacent DSI Registration Wand (Methods 2). Habituation was done in three 30 min blocks/day of 0.025 Hz, ±2 mA sGVS with 45 min of interspersed rest and test periods (Methods 3). If a VVR occurs, as shown by the drop in BP and HR, 30 min periods of rest were given before reinstituting the habituation stimulus (Methods 3). Susceptibility to habituation was tested in each 45 min rest period with a ±3 mA sGVS and a 70° nose-up tilt. They had 15 habituating sessions (3/day) over a 2 week period, which brought them into resistance to development of VVRs. If this did not occur, then they received an additional two weeks of habituation. During the entire process, BP, HR, baroreflex sensitivity (BRS) and recordings of low frequency oscillations (0.025 & 0.05 Hz) were determined (Methods 2, 4, 5). These data provided the basis for determining the ability to habituate, the duration and the nature of the process, whether it can be reinstated and the underlying mechanisms of habituation. Response to ±0.025, 3 mA sGVS and 70° nose-up tilts were collected to provide a comparative data base for susceptible and non-susceptible rats.

### Methods 1

### Surgical and Experimental Procedures

Twenty five, adult, male, Long-Evans rats (Harlan Laboratories, MA) weighing between 300 and 400 g were used in each year of this study. All experiments were approved by the IACUC of the Icahn School of Medicine at Mount Sinai. Based on our experience in the preceding grant period, most of the rats were initially susceptible to development of VVRs, and they had frequent VVRs when they are stimulated with sinusoidal Galvanic Vestibular Stimulation (sGVS), 70° tilts (0.91 g), and ±70° oscillation in pitch (±0.91 g). We called these, ‘susceptible rats’. Based on our experience, about 80% of rats were susceptible, so that about 5 rats were non-susceptible.

### Surgery

Surgery and all experiments were performed under Isoflurene anesthesia, 4% induction, 2% maintenance with oxygen. While in surgery and during experiments, the animals were kept on a temperature-controlled heating pad at 37° C. An IntraAortic Sensor (DSI, St. Paul, Minn.) was implanted in the abdominal aorta. Through an incision in the groin, the femoral artery was isolated and clamped. The transducer catheter was inserted into the vessel via a small arteriotomy and advanced into the abdominal aorta. The catheter was secured with ties around the artery and the body of the sensor was placed into a subcutaneous pocket in the flank. Adequate pain medications insured that the animals did not suffer post-operative pain.

### Experimental Protocol

The sGVS is generated by a computer-controlled stimulator. Currents of ±2 and ±3 mA at frequencies of 0.025 and 0.05 Hz were delivered via sub-dermal needle electrodes placed into the skin over the mastoids. These currents and frequencies were the most provocative for inducing VVRs and habituation, and were used in all of the habituation and test procedures. Rats were tilted 70° nose-up (0.91 g) and oscillated ±70° (±0.91 g) in pitch on a computer controlled platform. The axis of rotation was 13 cm from the head of the rat, activating both the otoliths and vertical semicircular canals. During the tilt and pitch experiments, rats were enclosed in a plastic box with soft packing material that stabilized the head and body so that the pitch occurred around the pitch axis. The position of the tilt table was recorded. During nose-up tilts to test for VVR sensitivity, the rats were left in the tilted position for 5 min if a VVR was not induced before being brought back to the prone position.

### BP and HR Measurement

Intra-aortic BP was transduced by a telemetric sensor in a wand receiver (DSI, St Paul, Minn.) placed close to the rats. Recordings of BP, as well as the position of the tilt table and the current levels of sGVS were sampled at 1 kHz with 12 bit resolution (Data Translation, Inc., MA). The BP and HR were continuously monitored and recorded during these experiments. Heart rate was computed offline from the systoles, which occurred on average at about 300 per min., giving a sample rate of BP of 200 msec per sample. Average systolic, diastolic, and mean BPs were computed, but had no significant differences on average, so systolic BP was used.

### Methods 2

### Data Collection

BP data from the telemetric sensors were collected via a wand receiver (DSI) BP, PPG and breathing rate sensor data as well as position of each axes and GVS current level were sampled at 1 KHz with 12 bit resolution (Data Translation, Inc.) using our in house developed data collection program. The data was converted for analysis using our VMF data analysis software. Signals derived from the intra-aortic BP sensor were used to compute BP and HR.

### Methods 3

### Habituation With sGVS and Oscillation in Pitch

Susceptible rats were habituated each day with either 30 min periods of ±2mA, 0.025 Hz sGVS (sGVS rats, Aim 1) or ±70° oscillation in pitch (Pitch rats, Experiments 2 and 3). The sGVS rats lay prone on a heating pad and the rats that were pitched will lay on the oscillating platform with their heads 13 cm from the center of oscillation. Thus, both the vertical semicircular canals and otoliths were activated by the pitch stimulus. Both sGVS and pitch rats were tested with ±3 mA, 0.025 Hz sGVS and a 70° nose-up tilt after each period of habituation. Each experimental session began with a 45 min test period, followed by the three alternate habituation and test periods for a minimum total experimental time of 5 hr 30 min on each day. The animals were interleaved, so that they were habituated and tested either two or three times a week, and had a day of rest after each test day, as well as an extra two rest days on weekends. Following each habituation period when the animals were tested for vasovagal oscillations (VVOs) or VVRs. 15 min were allowed to elapse between subsequent tests if there was no response to either the sGVS or to nose-up tilt. If a VVR or a partial VVR was induced, as detected by a fall in BP and HR, 30 min were allowed to elapse before the next test. Overall, the animals had 15 exposures to 30 min of habituation in five days over two weeks. If habituation had not been attained, they received additional training sessions until habituation was achieved.

### Methods 4

### Wavelet Analysis

Wavelet transforms were utilized to assess the temporal changes in BP and HR oscillations for specific frequency bands or scales. A complete description of the analysis was given in Cohen, B., et al., Sinusoidal galvanic vestibular stimulation (sGVS) induces a vasovagal response in the rat. Exp Brain Res, 2011. 210 (1): p. 45-55, which is hereby incorporated by reference. The analysis was performed using Matlab (Mathworks, Inc.) and its implementation of the Daubechies function, db12, the mother wavelet. This high order filter allowed the capture of dominant frequency components of the entire transient signal that was present in both BP and HR at the onset of the VVR, as well as the higher frequency bands. Analysis showed that the sum of 12 decomposed signals in different bands were equivalent to the original data and therefore temporal variations associated with the transient component and those associated with VVOs could be either individually analyzed or were summated to examine the composite waveform. Typically 5 min of data that included a pre-stimulus, stimulation, and post-stimulus period were processed with wavelet analysis. The power distribution of the waveforms in wavelet band numbers 10 (0.05 Hz), 11 (0.025 Hz) and an approximation band (12), that reflected the Transient response, i.e., the joint fall in BP and HR at the onset of the VVR, were determined and compared with the stimulus distribution. From this, we obtained how power in the band containing the stimulus frequency was related to those distributed throughout other bands and what role it played in initiating the VVR. To obtain the power of each band, the activity was squared and averaged.

### Methods 5

### Calculation of BaroReflex Sensitivity (BRS)

A period of 20 s of BP data before the onset of the sGVS was used. A peak finding algorithm identified each systolic/diastolic cycle (See Method 1). The time durations tᵢ between two systolic peaks (systBPᵢ and systBPᵢ₊₁), termed Intersystolic Interval, were plotted against the first systolic peaks (systBPᵢ). The slope of the linear regression was defined as the baroreflex sensitivity, which was the ratio between the change of Intersystolic interval and the change of systolic BP.

### Statistical Analysis

It was not possible to compare longitudinally the baroreflex sensitivity (BRS) of habituated animals with those that were not susceptible, as we did not have equal and large numbers of rats and the power of such an experimental design was low. Also each rat was different and had a different BRS. Statistical analysis was done by examining the BRS at different habituated states. The Null hypothesis was that the mean BSR in the habituated state was equal to the BSR of non-susceptible animals. Therefore, a large number of repeated tests in non-susceptible animals and susceptible animals gave us sufficient power to say that a t-test that cannot reject the Null hypothesis confirmed the hypothesis. The power was large because of the number of habituated states that were examined and so the number of animals did not have to be large. The statistical package within Matlab was used to determine whether the time course of habituation significantly modified the BRS. There were functions in the Statistical Package that could compute an F-test, t-test, and Welch-test. BRS was plotted as a function of time of habituation and was evaluated to determine whether the BRS in a habituated state at a specific time generated significantly different mean values of the BRS than before. At each level of the habituated state, an F-test or a Welch test was performed on the ratio of the two variances at the different times to determine if they were significantly different.

According to embodiments of the present disclosure, resistance to vasovagal response can be imparted in a patient (i.e., human patent) according to very similar methods to those discussed above with respect to the animal model. In at least one embodiment, resistance to the vasovagal response (i.e., resistance to unexpected fainting or syncope) can be achieved via induced galvanic vestibular stimulation of the patient. This resistance can be imparted by stimulating the vestibular system in general and the vestibular otolith in particular, via transmission of current through the vasovagal region carrying a repeating or periodic signal. For example, according to some embodiments, the induced galvanic vestibular stimulation comprises applying an alternating current to the patient such that the alternating current stimulates a vestibular otolith of the patient.

Inducing the galvanic vestibular stimulation can include applying a repeating waveform at a treatment amplitude in the range of 0.4 to 2 mA, or in some cases, from 0.1 to 4 mA. In some specific embodiments, the treatment amplitude can be approximately 2 mA. According to some embodiments, the repeating waveform can have a frequency in the range of 0.01 Hz to 0.5 Hz, or from 0.01 to 0.1 Hz, or in some specific cases, of approximately 0.025 Hz. The specific type of repeating signal can include, e.g., a sinusoidal signal or other comparable, periodic signal.

A treatment program can include providing multiple treatments to a patient over the course of one or more treatment sessions. According to some embodiments, a treatment program includes at least two treatment sessions. A single treatment session can include treating the patient with the induced galvanic vestibular stimulation for a therapeutic treatment period sufficient to induce a reduction in the vasovagal response. According to some embodiments, a therapeutic treatment period is in the range of about 15 to 30 minutes, or from about 20 to 30 minutes. According to some embodiments, a ramp period is added to the treatment session, the ramp period including a short duration during which the amplitude of the applied stimulation is gradually increased from a low value (e.g., 0 mA, or any suitable value less than the treatment amplitude) to the treatment amplitude. According to some embodiments, the ramp period has a duration of at least 200 seconds, or in some cases, in the range of 200 to 300 seconds.

### System

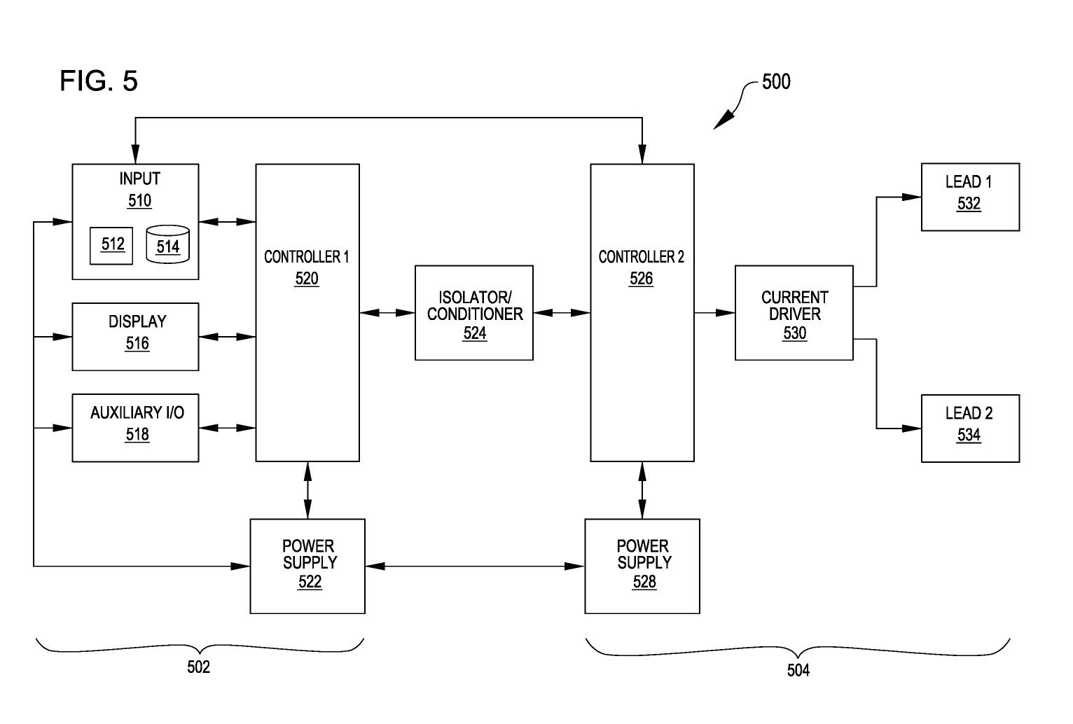

Resistance to vasovagal response can be induced in a patient by galvanic stimulation using a system similar to the system 500 illustrated in FIG. 5, in accordance with at least one embodiment of the present disclosure. The system 500 includes an input portion 502 and an output portion 504 for administering vestibular stimulation to a patient. The input portion 502 includes an input device 510 for generating the desired parameters of a repeating signal for application to the patient, and a first controller 520 for generating the repeating signal based on the parameters. The input device 510 can be, e.g., a computer or comparable device, such as a tablet, smartphone, control console, or other suitable input device. The input device 510 can include a processor 512 and nontransitory memory 514, which can be used to contain and implement executable instructions to generate the repeating signal, or to run a program for generating the repeating signal according to a pattern, including durations of a ramp and/or treatment period, amplitudes, frequencies, and the specific waveform pattern.

The system 500 can also include a display 516 operably connected with one or both of the input device 510 and the first controller 520, and may include a display 516 for displaying parameters to a user, and an auxiliary input/output device 518 for connecting the controller with power supply 522, with the input device, with the display, or to provide additional controls for varying the various parameters of the stimulation. According to some embodiments, the input device 510 and first controller 520, display 516, auxiliary I/O device 518, and/or power supply 522 may be connected together, e.g. by way of a universal serial bus connector (USB). According to some embodiments, the first controller 520 can provide a personal computer (PC) communication protocol for interfacing with the input device 510. According to some embodiments, the first controller 520 is a microcontroller.

The output portion 504 of the system 500 includes a second controller 526 (or microcontroller) which is powered by a power supply 528 and generates the required waveform for output to a current driver 530 that ultimately outputs the desired stimulation to the patient via a pair of leads 532, 534. The second controller 526 receives as input a signal from the first controller 520, which is filtered through an isolator/conditioner element 524. According to some embodiments, the isolator/conditioner 524 is simply an isolation device that prevents direct transmission of high amplitudes to the second controller 526, thereby acting as a safety device to protect the patient. According to some specific embodiments, the isolator/conditioner 524 is an optical isolator; however, in alternate embodiments, the isolator/conditioner can include any other suitable isolator or signal conditioning circuit, including but not limited to a digital isolator, magnetic isolator, capacitive isolator, filter, or the like to restrict the amplitude and/or frequency to a suitable range.

In some embodiments, the first power supply 522 and second power supply 528 can be separate, i.e., isolated from one another; but in some other embodiments, they can be the same power supply. In alternative embodiments, the system 500 may operate without certain elements of the input portion 502, e.g., the system 500 may operate in a minimal configuration in which the second controller 526, current driver 530, and power supply 528 can operate as a standalone device; or in conjunction with the input device 510, without connecting to an intermediate first controller 520 and isolator/conditioner 524.

According to at least one embodiment, the system 500 includes executable instructions stored at one of the input device 510 or first or second controller 520, 526 that cause the system to generate a repetitive waveform for application to a patient. The repetitive waveform is passed to the current driver 520, which drives the waveform to at least one pair of electrical leads 532, 534 (e.g. electrodes for connecting to a patient's skin). The leads 532, 534 impart the signal to the patient when the leads are connected to the patient at a treatment site corresponding with the vestibular system of the patient. According to some embodiments, the leads 532, 534 are configured to connect with the patent at a mastoidal site, and to impart the stimulation to the vestibular system via, e.g., the vestibular otolith. According to some embodiments, the system 500 is designed such that one, or both, of the controllers 520 are configured to generate the repetitive waveform at a frequency in the range of 0.01 to 0.05 Hz., or in some cases, from 0.01 to 0.1 Hz, or at about 0.025 Hz.

Other variations are within the spirit of the present invention. Thus, while the invention is susceptible to various modifications and alternative constructions, certain illustrated embodiments thereof are shown in the drawings and has been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

1.-20. (canceled)

21. A method of imparting resistance to vasovagal response in a patient, the method comprising:
inducing galvanic vestibular stimulation of the patient; and
repeating the inducing galvanic vestibular stimulation such that the patient is habituated to resist an induction of the vasovagal response.

22. The method of claim 21, wherein the inducing galvanic vestibular stimulation comprises applying an alternating current to the patient such that the alternating current stimulates a vestibular otolith of the patient.

23. The method of claim 21, wherein the inducing galvanic vestibular stimulation comprises applying a repeating waveform at a treatment amplitude in the range of 0.4 to 2 mA.

24. The method of claim 21, wherein the inducing galvanic vestibular stimulation comprises applying a repeating waveform having a frequency in the range of 0.01 Hz to 0.05 Hz.

25. The method of claim 21, wherein the inducing galvanic vestibular stimulation comprises applying a sinusoidal waveform.

26. The method of claim 21, wherein the inducing galvanic vestibular stimulation comprises applying the stimulation for at least one period of time corresponding to 15 to 30 minutes.

27. The method of claim 21, wherein the inducing galvanic vestibular stimulation comprises applying the stimulation for a first ramp period and a second treatment period after the ramp period, wherein the stimulation ramps from an amplitude of 0 to a treatment amplitude that is greater than zero during the ramp period, and wherein the stimulation is induced at the treatment amplitude for the second treatment period.

28. The method of claim 27, wherein the ramp period has a duration of at least 200 seconds.

29. The method of claim 21, wherein the vasovagal response comprises unexpected syncope.

10. The method of claim 21, wherein the inducing galvanic vestibular stimulation comprises applying the stimulation via one or more electrodes at a mastoidal site of the patient such that the stimulation is applied to a vestibular otolith.

11. A system for imparting resistance to vasovagal response in a patient by galvanic stimulation, the system comprising:
a controller comprising a processor and nontransitory memory containing executable instructions that, when executed by the processor, cause the controller to generate a repetitive waveform;
a current driver operably connected with the controller and configured to generate a signal suitable for application to a patient based on the waveform; and
at least one pair of electrical leads operably connected with the current driver and configured to impart the signal to the patient when the pair of electrical leads is connected to the patient at a treatment site corresponding with the vestibular system of the patient.

12. The system of claim 31, further comprising:
an isolation element operably connected between the controller and the current driver.

13. The system of claim 12, wherein the controller is a first controller and the waveform is a first waveform, and further comprising:
a second controller operably connected between the isolation element and the current driver, the second controller configured to generate a second waveform based on the first waveform filtered by the isolation element, wherein the current driver is configured to generate the signal suitable for application to a patient based on the second waveform.

14. The system of claim 31, wherein the controller is further configured to generate the repetitive waveform at a frequency in the range of 0.01 to 0.05 Hz.

15. The system of claim 31, wherein the controller is further configured to generate the repetitive waveform at a frequency of about 0.025 Hz.

16. The system of claim 31, wherein the repetitive waveform is a sinusoidal waveform.

17. The system of claim 31, wherein the controller is further configured to generate the waveform at an increasing amplitude for a first nonzero duration of a ramp period during which the amplitude increases from an initial amplitude to a treatment amplitude, and to generate the waveform at the treatment amplitude for a second nonzero duration of a treatment period subsequent to the ramp period.

18. The system of claim 37, wherein the first duration is at least 5 minutes, and wherein the second duration is in the range of 20 to 30 minutes.

19. The system of claim 31, wherein the at least one pair of electrical leads comprises electrodes configured to attach to the skin of a patient proximate to a mastoidal site.

20. The system of claim 31, wherein the at least one pair of electrical leads is configured to stimulate the vestibular otolith of the patient.
